(19) United States
(12) Patent Application Publication
(10) Pub. No.: US 2007/0142813 A1
(43) Pub. Date: Jun. 21, 2007

(54) Disposable garment with regulated friction feature

(75) Inventor: Michael D. Sperl, Waupaca, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/303,218

(22) Filed: Dec. 15, 2005

(51) Int. Cl. A61F 13/15

(52) U.S. Cl. 604/385.31; 604/385.24

(57) ABSTRACT

A garment with a gasket flap that exposes an ever increasing area of higher coefficient of friction or grip zone in response to an increased load in the garment due to body exudates or the like. The grip zone has a coefficient of friction that is greater than the portion of the gasket flap initially exposed to the wearer's skin. The gasket flap may be positioned at various places in the garment to prevent slipping or the like.

### BACKGROUND OF THE INVENTION

This invention relates generally to a disposable garment for personal wear, and more particularly to such a disposable garment with a gasket feature to maintain a desired position of the garment on the wearer.

Disposable garments are known for use in the manner of underpants for children and adults, garments worn like training pants for toddlers and garments worn like diapers for infants. Disposable absorbent garments are designed to absorb and contain bodily fluids but otherwise have a limited period of use before its ability to perform its intended function is exhausted. In particular, such disposable absorbent garments are intended to be discarded after one or more insults instead of being laundered.

Typically, it is desirable for disposable garments to fit close to the body of the wearer for comfort and discretion. Presently available disposable garments for wear about one's waist include a substantially liquid impermeable outer cover, a liquid permeable bodyside liner in a superposed relation with the outer cover, and an absorbent structure located between the outer cover and the bodyside liner for taking in and retaining liquid body exudates. The side edges of the garment form a pair of leg openings when the disposable garment is worn and the ends of the garment together form the waist opening thereof. Elastic components such as waist elastics, leg elastics and containment flaps are commonly used to enhance the fit and inhibit leakage from the garment.

Absorbent articles in the form of garments that are pre-assembled for slip-on application on the body of a wearer (e.g., training pants or pull-on diapers). In order both to contain bodily exudates and also to fit a wide variety of body shapes and sizes, such garments must fit snugly about the waist and legs of the wearer without drooping, sagging, or sliding down from their position on the lower torso, and without causing unnecessary pressure on the skin by reason of the product being too tight for the wearer's comfort.

Some newer disposable, absorbent garments incorporate an improvement such as an extensible or stretchable chassis. These garments are generally held in position on the body of the wearer by an elasticized structure that is positioned in the waist area of the garment. The elasticized structure is preferably under tension to generate a hoop stress within the waist structure and cause it to engage with and to press against the waist area of the wearer.

Gravity acting on the garment, movements by the wearer, and/or an increase in the weight of the absorbent exudate-receiving core resulting from the absorption and containment of bodily waste, may cause the garment to become displaced on the wearer and adversely affect fit.

In providing sufficient hoop stress within the elasticized waist structure to attempt to cause the garment to be retained in its initial position on the wearer's body, the inward force acting against the wearer's waist may causes pressure and tightness to be exerted on the body of the wearer, which in excess may cause wearer discomfort and/or undesirable pressure marks, sometimes referred to as “red marks,” on the wearer's skin about the wearer's waist. Such red marks are indicative of the relatively high inward forces that are imposed on the wearer's waist, and while they are harmless, they are undesirable both because they cause discomfort to the wearer and also because they cause anxiety to mothers of small children who wear such garments. The present invention is directed to minimizing discomfort and the attendant red marking of the wearer's skin by providing increased surface friction between the garment and the wearer's skin, which enables the hoop stress, and the resulting inward forces acting against the wearer's body, to be reduced.

The broad notion of increasing the coefficient of friction of an interior surface of a disposable absorbent article is known. For example, it is taught that a friction agent may be placed at the hip or waist portions of the article. However, it is desirable to have the friction agent available as needed so that softer and more comfortable materials contact the wearer's skin as long as possible during a given period of wear. It is also known to place a cohesive at the back portion of the diaper so that it adheres to the skin of the wearer. However, cohesive may not allow as much freedom of use as it is difficult for the wearer to apply to him or herself and must be disengaged from the skin if the wearer desires to pull the garment down.

Thus, even though significant and numerous advancements have occurred in the materials and structural features available for the construction of disposable garments, there remain opportunities for improvement in the fit of such garments. For example, there remains a need for a disposable garment that provides improved fit on the wearer while inhibiting sagging and red marks about the wearer's waist.

### SUMMARY OF THE INVENTION

The present invention is a disposable garment adapted to be worn about a lower torso of a body of a wearer. In one aspect of the invention, a chassis includes leg elastics defining a pair of leg openings, and a waistband defining a waist opening. These are collectively referred to as garment openings. A gasket flap located at one of the plurality of openings. The gasket flap includes a body-facing surface having a first coefficient of friction and an inner surface that has a second coefficient of friction different from that of the first surface. In addition, the gasket flap has a first end and a second end bonded to the garment with a graduated bond to increase resistance against roll back of the gasket flap as more of the inner surface is exposed.

In another aspect of the invention a garment includes a chassis having a front region, a back region, a crotch region positioned between the front region and the back region, and a waist region formed at the upper portion of the front region and the back region. A pair of side panels overlie a hip area of the wearer and thereby form a pair of laterally spaced leg openings and a waistband defining a waist opening spaced from each of the leg openings. The leg openings and the waist opening are collectively referred to as a plurality of garment openings. A gasket flap is joined to the garment at one of the plurality openings. The gasket flap includes a body-facing surface having a first coefficient of friction and an inner surface that has a second coefficient of friction different from that of the first surface. In addition, the gasket flap has a first end and a second end bonded to the garment with a graduated bond to increase resistance against roll back of the gasket flap as more of the inner surface is exposed.

Other features will be in part apparent and in part pointed out hereinafter.

### BRIEF DESCRIPTION OF THE DRAWINGS

FIG. 1 is a perspective view of a disposable garment of the present invention in a partially fastened condition;

FIG. 2 is an outer plan view of a disposable garment of FIG. 1 in the form of a diaper illustrated in an unfolded and laid flat condition to show the surface of the diaper that contacts the skin of the wearer;

FIG. 3 is an inner plan view similar to FIG. 1 but with portions cut away to reveal internal construction;

FIG. 3a is a partial close-up of the regulated bond region of the garment of FIG. 3;

FIG. 4 is a partial cross-sectional side elevation of the garment shown in FIG. 2, taken at lines 4-4, showing the waist flap in a bonded state;

FIG. 5 is a cross-sectional side elevation of the waist flap shown in FIG. 4, it a detached state;

FIG. 6(a-d) is schematic illustration of the regulated bond region of the garment in FIG. 3, in various stages of detachment;

FIG. 7 is a partial perspective view of a pair of slits on an edge of the gasket flap of the present invention used to encourage roll back; and

FIG. 8 is a side elevation of another embodiment a fastened garment of the present invention, showing several different possible gasket flap locations on the garment of FIG. 1.

Corresponding reference characters indicate corresponding parts throughout the several views of the drawings.

### DETAILED DESCRIPTION OF THE INVENTION

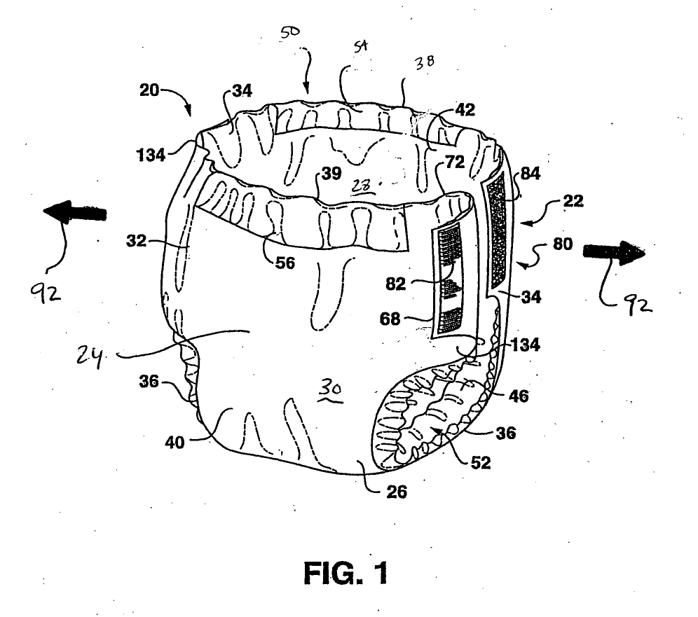

Referring to FIG. 1, for exemplary purposes, an absorbent article 20 that may be made in accordance with the present invention is shown. The absorbent article 20 may or may not be disposable, which refers to articles that are intended to be discarded after a limited period of use instead of being laundered or otherwise conditioned for reuse. It is understood that the present invention is suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing disposable absorbent article such as the pant 20 of the various aspects of the present invention may be disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al.; which are incorporated herein by reference to the extent they are consistent (i.e., not in conflict) herewith. However, it is contemplated that the regulated friction feature of the present invention could be incorporated into a diaper or an adult or feminine care garment.

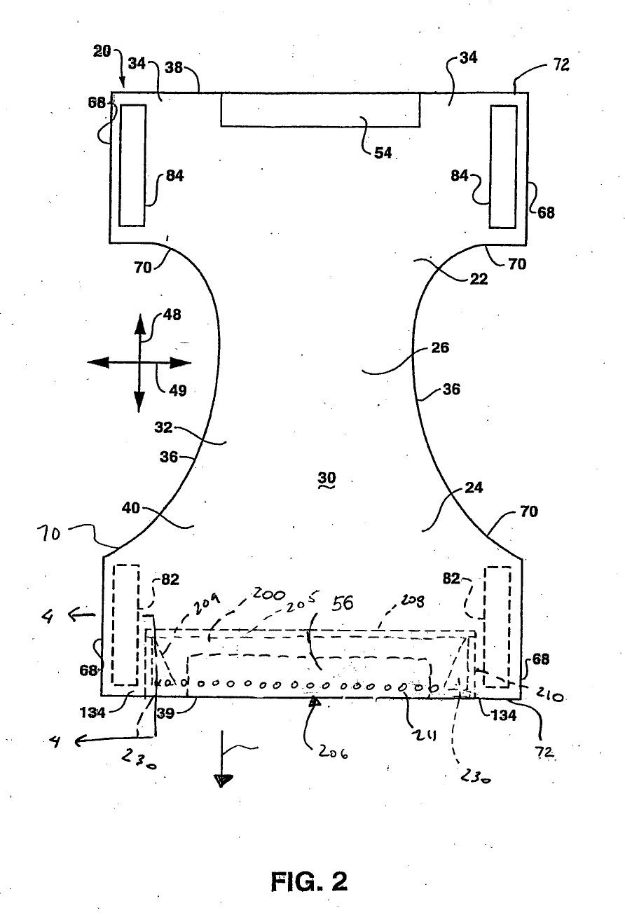

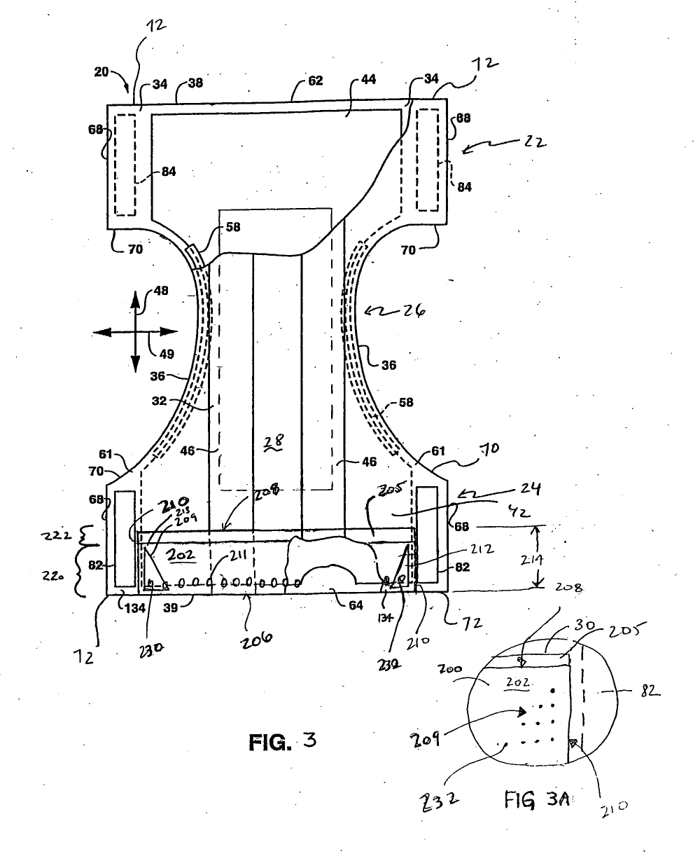

A pair of training pants 20 is representatively illustrated in FIG. 1 in a partially fastened condition. The training pants 20 shown in FIG. 1 is also represented in FIGS. 2 and 3 in an opened and unfolded state. Specifically, FIG. 2 is a plan view illustrating the exterior side of the pants 20, while FIG. 3 illustrates the interior side of the pants 20. As shown in FIGS. 2 and 3, the pant 20 defines a longitudinal direction 48 that extends from the front of the training pants when worn to the back of the training pants. Opposite to the longitudinal direction 48 is a lateral direction 49.

The pants 20 define a pair of longitudinal end regions, otherwise referred to herein as a front region 22 and a back region 24, and a center region, otherwise referred to herein as a crotch region 26, extending longitudinally between and interconnecting the front and back regions 22, 24. The pant 20 also defines an inner surface 28 adapted in use (e.g., positioned relative to the other components of the pants 20) to be disposed toward the wearer, and an outer surface 30 opposite the inner surface. The front and back regions 22, 24 are those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The training pants 20 have a pair of laterally opposite side edges 36 and a pair of longitudinally opposite waist edges, respectively designated front waist edge 38 and back waist edge 39.

The illustrated pants 20 may include a chassis 32, a pair of laterally opposite front side panels 34 extending laterally outward at the front region 22 and a pair of laterally opposite back side panels 134 extending laterally outward at the back region 24.

Referring to FIGS. 1-3, the chassis 32 includes an outer cover 40 and a bodyside liner 42 (FIGS. 1 and 3) that may be joined to the outer cover 40 in a superimposed relation therewith by adhesives, ultrasonic bonds, thermal bonds or other conventional techniques. Referring to FIG. 3, the liner 42 may suitably be joined to the outer cover 40 along the perimeter of the chassis 32 to form a front waist seam 62 and a back waist seam 64. As shown in FIG. 3, the liner 42 may suitably be joined to the outer cover 40 to form a pair of side seams 61 in the front region 22 and the back region 24. The liner 42 can be generally adapted, i.e., positioned relative to the other components of the pants 20, to be disposed toward the wearer's skin during wear of the pants.

The chassis 32 may further include an absorbent structure 44 particularly shown in FIG. 3 disposed between the outer cover 40 and the bodyside liner 42 for absorbing liquid body exudates exuded by the wearer, and may further include a pair of containment flaps 46 secured to the bodyside liner 42 for inhibiting the lateral flow of body exudates.

With the training pants 20 in the fastened position (as demonstrated by the partially fastened in FIG. 1), the front and back side panels 34,134 can be connected together by a permanent bond (not shown) or a fastening system 80 to define a three-dimensional pants configuration having a waist opening 50 and a pair of leg openings 52. The front and back side panels 34 and 134, upon wearing of the pants 20, thus include the portions of the training pants 20 which are positioned on the hips of the wearer. The waist edges 38 and 39 of the training pants 20 are configured to encircle the waist of the wearer and together define a waist opening 50 of the pants.

The elasticized containment flaps 46 as shown in FIG. 3 define a partially unattached edge which assumes an upright configuration in at least the crotch region 26 of the training pants 20 to form a seal against the wearer's body. The containment flaps 46 can extend longitudinally along the entire length of the chassis 32 or may extend only partially along the length of the chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pants 20 may also suitably include a front waist elastic member 54 (FIG. 1), and/or a rear waist elastic member 56, and leg elastic members 58 (FIG. 3), as are known to those skilled in the art. The waist elastic members 54 and 56 can be operatively joined to the outer cover 40 and/or the bodyside liner 42 and can extend over part or all of the waist edges 38, 39. The leg elastic members 58 can be operatively joined to the outer cover 40 and/or the bodyside liner 42 and positioned in the crotch region 26 of the training pants 20.

The waist elastic members 54 and 56, and the leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate. In one particular aspect, for example, the leg elastic members 58 may include a plurality of dry-spun coalesced multifilament spandex elastomeric threads sold under the trade name LYCRA and available from Invista, Wilmington, Del., U.S.A.

In one embodiment of the present invention, a gasket flap 200 is placed lieu of a waist elastic member or in addition thereto, either on the back region 24 adjacent waist end edges 72, and/or the front region 22. Gasket flap 200 has an outer surface 202 that initially, is a bodyfacing surface. Gasket flap 200 also has an inner surface 204 that initially faces, and suitably may even contact, liner 42. Gasket flap 200 may be rectangular in shape, and defined by a waist edge 206, a interior edge 208, and a pair of opposite side edges 210. However, other shapes may advantageous, for example, a shape having a convex or concave interior edge 208 (not shown). Gasket flap 200 may be placed on other portions of the garment as described herein.

Suitably, and in particular if the gasket flap 200 may replace waist elastic member 54 or 56, the gasket flap 200 may be made from various stretchable materials such as single-faced spun bond laminate (SBL), and the like. The waist flap outer surface 202 has a lower coefficient of friction (COF) than its inner surface 204. The outer surface 202 may slide easily across that wearer's skin, and inner surface 204 slide much less easily across the skin, if at all. The differences between the skin of various wearer's may affect whether or not the surface 202 slides across the skin during wear. Regardless, with this arrangement, exposure of inner surface against the wearer's skin may have the desired result of preventing the garment from drooping or sagging down from the wearer's waist region.

Gasket flap 200 is attached to a waist region, such as the back region 24, as follows. The waist edge 206, or proximate region thereto, may be continuously or partially attached to the back region adjacent to waist edge 72. For example, the waist edge attachment may be made with a relatively permanent bond, such as the series of bonds 211. The bond(s) may be continuous or discontinuous discrete bonds made by ultrasonic, pressure adhesive, thermal, stitch or other methods known in the art. The bonds used to attach gasket flap 200 to the garment chassis may be used to commonly attach waist elastic 54 or 56 to the chassis. For example, the same set of bonds 211 may operate to attach waist elastic 56 and gasket flap 200 to the garment 20 as seen in FIG. 2.

Generally, flap side edges 210 are attached from about waist edge 206 to about one-third to about two thirds the distance 204 to the interior edge 208. The opposite interior edge 208 is left free. Because the interior edge 208 is free and side edges 210 are not completely attached to waist edge 206, as the back region 21 is extended, the interior edge 208, when stretched, tends to curl up to reveal inner surface 204; this curl is referred to as lip 205. The purpose of lip 205 is to provide some contact between the wearer's skin and the flap inner surface to encourage rolling of the flap 200 should downward movement of the garment at the waist region occur. The formation of lip 205 may due the natural curl of the waist flap interior edge 208, or it may be permanently set or pressed into that position during manufacture of the garment. For example, the curl may be produced by having a differential basis weight of facing material attached to opposite sides of the elastic member.

The inner surface 204 that is exposed to engage the wearer's skin may be referred to as the grip zone. The lip 205 has a predetermined area, whereas the grip zone may increase or decrease in area during use. Once the inner surface 204 or grip zone is exposed, it is possible for flap 200 to peel or roll back to the initial point where only the lip 205 is revealed. For example, this may happen if the garment is pulled up to or past the waist of the wearer. The grip zone may include an elastic film, elastomeric strands, a coating. More specifically, the grip zone or inner surface 204 may include materials as described in U.S. Pat. No. 6,135,988 incorporated herein to the extent that it is consistent with the present invention. Moreover, the inner surface 204 may include or have placed thereon: rubber-solvents, cellulosic, gums, minerals, polymers, resins and combinations therof, an elastic film, example PEBAX from AtoChem located in Philadelphia, Pa., elastomeric strands (example KRAYTON from Raton polymers of Houston, Tex. or LYCRA from Invista, Inc. Wilmington, Del.)., and/or a coating product Number 10958-17-1 from National Starch and Chemical, Inc. located in Bridgewater, N.J.

In a first embodiment as seen in FIGS. 2-5, the attachment of side edges 210 to garment waist region are such that interior edge 208 can be rolled back to reveal inner surface 204 up to a region of initial attachment 213 or 232. However, the region of initial attachment 213 can detach once acted upon by a predetermined load to expose the grip zone at inner surface 204.

In FIGS. 2 and 3, the attachment of side edges 210 is schematically defined by area 209, a triangular shape. However, the triangular shape is only meant to be a representation of the overall physical strength of the bond(s) forming the attachment of side edges 210. Regardless of whether area 209 is constructed with discrete bonds that vary in size, shape, and/or bond type; or a continuous bond such as an adhesive film or the like, area 209 is a “graduate bond.”

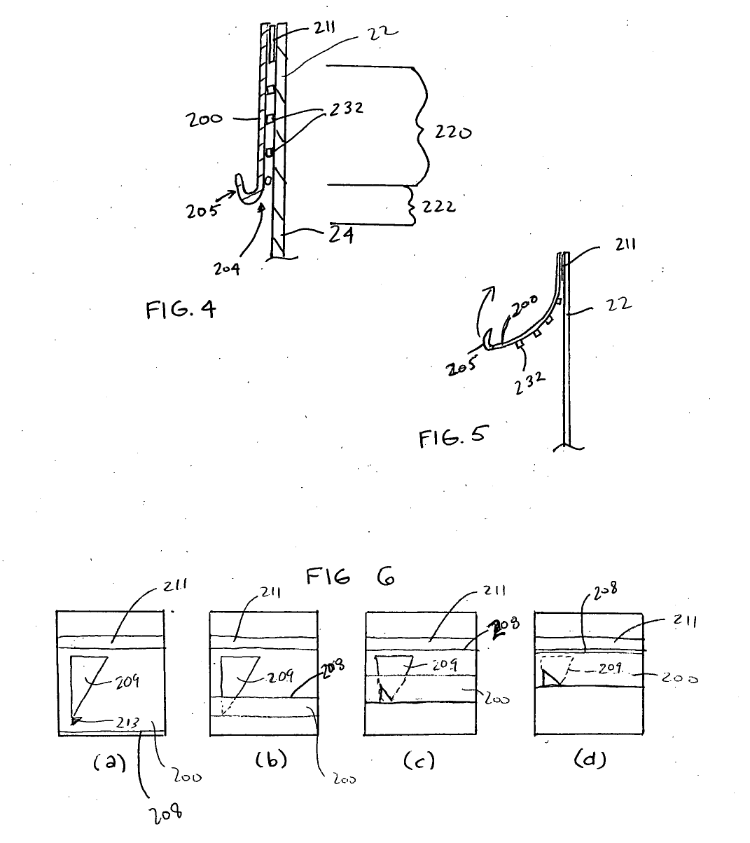

Specifically, within the graduated bond area 209, the bond strength, e.g. peel strength, may increase in the longitudinal direction toward waist edge 38 or 39. The attachment of side edges 210 to the garment 20 may be obtained with continuous or discontinuous, ultrasonic, adhesive, mechanical, pressure, or stitched bonds. Thus, if a continuous adhesive bond is used, the area 209 may indeed be triangular or the like so that the peel force increases as the gasket flap 200 is rolled back, provided there is enough force to roll/peel the waist flap as described below. If a discontinuous series of point bonds is used, the waist flap may roll/peel so as to break all bonds in a given area, up to where a relatively permanent bond is placed. As an example, in FIGS. 4-6 where a series of discrete bonds 232 are used to attach side edges 210 to garment 20, each discrete bond 232 or set of bonds 232 may become progressively resistant to peel forces as its position approaches relatively permanent bond 211. This progression to peel forces may be between each discrete bond 232, or even within a discrete bond 232. For example, if area 209 contained two discrete bonds, each of the discrete bonds 232 could have a non-uniform resistance to peel within the areas defining each of the discrete bonds 232.

In operation, the attachment bonds in area 209 may gradually detach as the garment load increases due to increased exudates or the like. For example, in FIG. 6(a), the gasket flap 200 is rolled back to the point of initial attachment 213, where in this example, there is only one bond point 232. Load on the garment 20 is increased, and the initial attachment 213 is broken. Gasket flap 200 rolls back until arrested by the second set of bond points 232, which is represented by two bond points in FIG. 6(b). Load on the garment 20 is increased, and the second set of bond points are broken. Gasket flap 200 rolls back until arrested by the third set of bond points 232, which is represented by three bond points in FIG. 6(c). Load on the garment 20 is increased, and the third set of bond points are broken. Gasket flap 200 rolls back until arrested by the fourth set of bond points 232, which is represented by four bond points in FIG. 6(d). If the load is increased again, the fourth set of bond points may break and the waist band 200 roll back until arrested by the relatively permanent bond point 211. This example is not meant to be limiting as the bonds points 232 may progressively increase in size, shape and/or bond strength instead of number. Further, the bond may be a continuous area that depends in shape to create an increase in bond strength.

In a second embodiment, the initial attachment 213 is meant to be relatively permanent, and is not broken by normal usage of the garment, such as by an increased load in or on garment 20.

In another embodiment of the present invention as seen in FIG. 8, the gasket flaps 200 may be arranged on the garment in a variety of ways. For example, the gasket flaps 200 may be in a parallel or stacked arrangement. In addition to or in the alternative, the gasket flaps 200 may be arranged in series. Suitably, gasket flaps 200 may be located on garment 20 in any portion of the bodyfacing surface.

Most suitably, the garment portions at which the gasket flaps 200 are positioned are those portions that are maintained against the wearer's skin, such as the waist region, side panels and leg elastics. Such portions of the garment structure are typically biased against the wearer's body to prevent leakage of bodily exudates. Gasket flaps 200 may vary in shape and/or size depending in location.

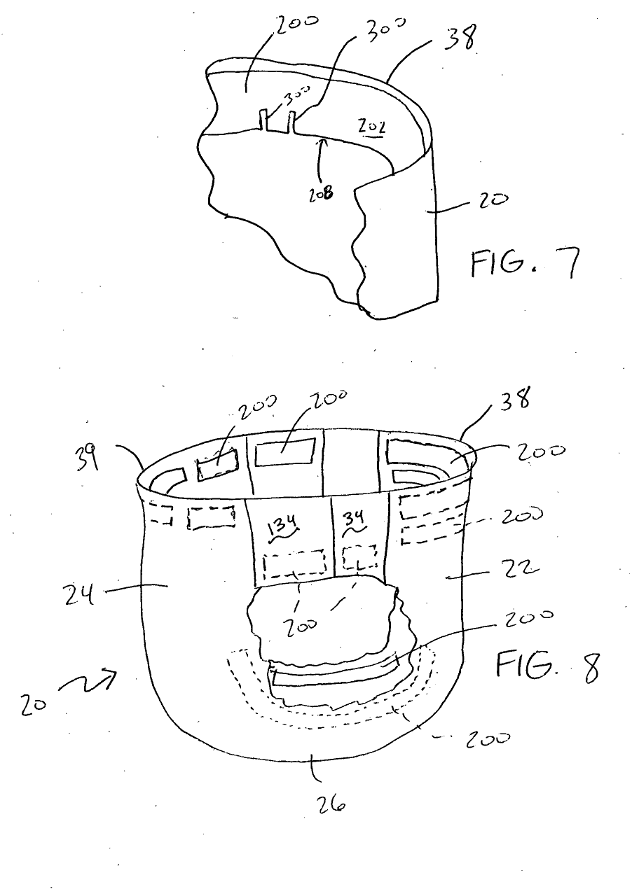

In yet another embodiment of the present invention as seen in FIG. 7, slits 300 may be cut into the free edge 208 of gasket flap 200 to encourage roll back and thus, grip zone exposure. Most suitably, the locations of the slits may be in areas of the gasket flap that maintain substantially constant contact with the wearer's skin. Pairs of slits 300 may be located along a select length of a gasket flap, or be presented as a single pair. Desirably, each slit 300 will not extend from edge 208 in the longitudinal direction past the point of initial attachment 213 as described previously.

As shown in FIGS. 1 through 3, the side panels 34 and 134 can be formed as an integral portion of the chassis 32. For example, the side panels 34, 134 can include a generally wider portion of the outer cover 40, the bodyside liner 42, and/or other components of the chassis 32. As described above, the side panels 34 and 134 may be attached together using any suitable fastening system 80.

In the embodiments shown in the figures, the side panels 34 and 134 are releasably attachable. It should be understood, however, that in other embodiments, the side panels 34 and 134 may be permanently joined together. For instance, the side panels may be made from a unitary piece of material. Alternatively, the side panels may be bonded together using ultrasonic bonding, thermal bonding or an adhesive. In this embodiment, the absorbent article is pulled over the legs when being worn.

In an alternative embodiment of the present invention (FIG. 8), the side panels 34 and 134 may be separately attached to the chassis 32. For instance, the front side panels 34 can be permanently bonded to and extend transversely outward beyond the side margins of the chassis 32. Similarly, the back side panels 134 can be permanently bonded to and extend transversely outward beyond the side margins of the chassis 32 and the back region 24. The side panels 34 and 134 may be bonded to the chassis 32 using attachment means known to those skilled in the art such as adhesive, thermal or ultrasonic bonding.

The front and back side panels 34 and 134 each have a longitudinal outer edge 68, and a leg end edge 70 disposed toward the longitudinal center of the training pants 20, and waist end edges 72 disposed toward a longitudinal end of the training pants. The leg end edges 70 and the outer edges 68 of the side panels 34 and 134 form part of the pant side edges 36 of the training pants 20. The leg end edges 70 of the absorbent article 20 may be suitably curved and/or angled relative to the lateral direction 49 to provide a better fit around the wearer's legs. However, it is understood that only one of the leg end edges 70 may be curved or angled, such as the leg end edge of the back region 24, or alternatively, neither of the leg end edges may be curved or angled, without departing from the scope of the present invention. The waist end edges 72 are suitably parallel to the transverse axis 49.

The waist end edges 72 of the front side panels 34 form part of the front waist edge 38 of the training pants 20, and the waist end edges 72 of the back side panels 134 form part of the back waist edge 39 of the pants. In the figures, the waist end edges 72 and the outer edges 68 are generally horizontal and vertical respectively. It should be understood, however, that in other embodiments, the waist end edges 72 and/or the outer edges 68 may have a curved, slanted or complex arrangement depending upon the particular application.

In configurations where the side panels 34,134 are separately attached (FIG. 8), the side panels may be provided by an elastic material capable of stretching at least in a direction generally parallel to the lateral direction 49 of the training pants 20. Suitable elastic materials, as well as one process of incorporating elastic side panels into training pants, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference to the extent that they are consistent (i.e., not in conflict) herewith. In particular aspects, the elastic material may include a stretch-thermal laminate (STL), a neck-bonded laminate (NBL), a reversibly necked laminate, or a stretch-bonded laminate (SBL) material. Alternatively, the side panel material may include other woven or non-woven materials, such as those described later herein as being suitable for construction of the outer cover 40 and/or the bodyside liner 42; mechanically pre-strained composites; or stretchable but inelastic materials.

The fastening system 80 may include laterally opposite first fastening components 82 adapted for refastenable engagement to corresponding second fastening components 84. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the pants 20 in its three-dimensional configuration.

The fastening components 82, 84 may be any refastenable fasteners suitable for absorbent articles, such as adhesive fasteners, cohesive fasteners, mechanical fasteners, or the like. In particular aspects the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 82 include loop fasteners and the second fastening components 84 include complementary hook fasteners. Alternatively, the first fastening components 82 may include hook fasteners and the second fastening components 84 may be complementary loop fasteners. In another aspect, the fastening components 82, 84 can be interlocking similar surface fasteners, or adhesive and cohesive fastening elements such as an adhesive fastener and an adhesive-receptive landing zone or material; or the like.

Although the training pants 20 illustrated in FIG. 1 indicate the back side panels 134 overlapping the front side panels 34 upon connection thereto, which is convenient, the training pants 20 can also be configured so that the front side panels 34 overlap the back side panels 134 when connected. One skilled in the art will recognize that the shape, density and polymer composition of the hooks and loops may be selected to obtain the desired level of engagement between the fastening components 82, 84. Optionally, either one or both of the fastening components 82, 84 may be provided by one of the inner or outer surfaces 28 and 30 of the side panels 34 and 134. Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

In the embodiment shown in FIG. 3, the absorbent article 20 further includes a surge management layer 60 which may be optionally located adjacent the liner 42 and/or the absorbent structure 44 and attached to various components in the article 20 such as the absorbent structure 44 or the bodyside liner 42 by methods known in the art, such as by using an adhesive. A surge management layer 60 helps to decelerate and diffuse surges or gushes of liquid that may be rapidly introduced into the absorbent structure of the article. Desirably, the surge management layer can rapidly accept and temporarily hold the liquid prior to releasing the liquid into the storage or retention portions of the absorbent structure. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

As described above, the present invention is particularly suitable for absorbent articles having controlled stretch properties. For example, the absorbent articles of the present invention may demonstrate biaxial stretch characteristics in both the lateral direction and the longitudinal direction, either everywhere or in selected areas of the garment. In one particular embodiment, the outer cover 40 and/or the bodyside liner 42 are made from biaxially stretchable and/or elastic materials. These materials are incorporated into the absorbent article 20 in a manner that provides the article with the desired stretch characteristics in the longitudinal direction and the lateral direction.

The outer cover 40, the inner liner 42 and the absorbent structure 44 may be made from many different materials. All three layers, for instance, may be stretchable and/or elastic. Further, the stretch properties of each layer may vary in order to control the overall stretch properties of the product.

The outer cover 40, for instance, may be breathable and/or may be liquid impermeable. The outer cover 40 may be constructed of a single layer, multiple layers, laminates, spunbond fabrics, films, meltblown fabrics, elastic netting, microporous webs, bonded card webs or foams provided by elastomeric or polymeric materials. The outer cover 40, for instance, can be a single layer of a liquid impermeable material, or alternatively can be a multi-layered laminate structure in which at least one of the layers is liquid impermeable. In other embodiments, however, it should be understood that the outer cover may be liquid permeable. In this embodiment, for instance, the absorbent article may contain an interior liquid barrier layer.

For instance, the outer cover 40 can include a liquid permeable outer layer and a liquid impermeable inner layer that are suitably joined together by a laminate adhesive, ultrasonic bonds, thermal bonds, or the like. Suitable laminate adhesives, which can be applied continuously or intermittently as beads, a spray, parallel swirls, or the like, can be obtained from Bostik Findley Adhesives, Inc., of Wauwatosa, Wis., U.S.A., or from National Starch and Chemical Company, Bridgewater, N.J. U.S.A. The liquid permeable outer layer can be any suitable material and is desirably one that provides a generally cloth-like texture. One example of such a material is a 20 gsm (grams per square meter) spunbond polypropylene nonwoven web. The outer layer may also be made of those materials of which the liquid permeable bodyside liner 42 is made.

The inner layer of the outer cover 40 can be both liquid and vapor impermeable, or it may be liquid impermeable and vapor permeable. The inner layer can be manufactured from a thin plastic film, although other flexible liquid impermeable materials may also be used. The inner layer, or the liquid impermeable outer cover 40 when a single layer, prevents waste material from wetting articles, such as bed sheets and clothing, as well as the wearer and caregiver. A suitable liquid impermeable film for use as a liquid impermeable inner layer, or a single layer liquid impermeable outer cover 40, is a 0.02 millimeter polyethylene film commercially available from Pliant Corporation of Schaumburg, Ill., U.S.A.

In most embodiments, the outer cover 40 is biaxially stretchable and optionally biaxially elastic. Elastic non-woven laminate webs that can be used as the outer cover 40 include a non-woven material joined to one or more gatherable non-woven webs, films, or foams. Stretch Bonded Laminates (SBL) and Neck Bonded Laminates (NBL) are examples of elastomeric composites. Non-woven fabrics are any web of material which has been formed without the use of textile weaving processes which produce a structure of individual fibers that are interconnected in an integrating manner.

Examples of suitable materials are spunbond-meltblown fabrics, spunbond-meltblown-spunbond fabrics, spunbond fabrics, or laminates of such fabrics with films, foams, or other nonwoven webs. Elastomeric materials may include cast or blown films, foams, meltblown fabrics or spunbond fabrics composed of polyethylene, polypropylene, or polyolefin elastomers, as well as combinations thereof. The outer cover 40 may include materials that have elastomeric properties through a mechanical process, printing process, heating process, or chemical treatment. For examples such materials may be apertured, creped, neck-stretched, heat activated, embossed, and micro-strained; and may be in the form of films, webs, and laminates.

Examples of a suitable material for a biaxially stretchable outer cover 40 is a breathable elastic film/nonwoven laminate, described in U.S. Pat. No. 5,883,028, issued to Morman et al., incorporated herein by reference to the extent that it is consistent (i.e. not in conflict) herewith. Examples of materials having two-way stretchability and retractability are disclosed in U.S. Pat. No. 5,116,662 issued to Morman and U.S. Pat. No. 5,114,781 issued to Morman, both of which are hereby incorporated herein by reference to the extent that it is consistent (i.e., not in conflict) herewith. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent structure 44. A suitable bodyside liner 42 may be manufactured from a wide selection of web materials, such as porous foams, reticulated foams, apertured plastic films, woven and non-woven webs, or a combination of any such materials. For example, the bodyside liner 42 may include a meltblown web, a spunbonded web, or a bonded-carded-web composed of natural fibers, synthetic fibers or combinations thereof. The bodyside liner 42 may be composed of a substantially hydrophobic material, and the hydrophobic material may optionally be treated with a surfactant or otherwise processed to impart a desired level of wettability and hydrophilicity.

The bodyside liner 42 may also be stretchable, and more suitably it may be elastomeric. Suitable elastomeric materials for construction of the bodyside liner 42 can include elastic strands, LYCRA elastics, cast or blown elastic films, nonwoven elastic webs, meltblown or spunbond elastomeric fibrous webs, as well as combinations thereof.

The bodyside liner 42 can also be made from extensible materials as are described in U.S. Pat. No. 6,552,245 filed on May 3, 2000 by Roessler et al which is incorporated herein by reference. The bodyside liner 42 can also be made from biaxially stretchable materials as are described in U.S. Pat. No. 6,641,134 filed on Oct. 27, 2000 by Vukos et al; which is incorporated herein by reference.

The absorbent structure 44 may be disposed between the outer cover 40 and the bodyside liner 42. The absorbent structure 44 can be any structure or combination of components which are generally compressible, conformable, non-irritating to a wearer's skin, and capable of absorbing and retaining liquids and certain body wastes. For example, the absorbent structure 44 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular aspect, the absorbent web material is a matrix of cellulosic fluff and superabsorbent hydrogel-forming particles. The cellulosic fluff may include a blend of wood pulp fluff. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art. Furthermore, the absorbent structure may itself encompass multiple layers in the Z direction. Such multiple layers may take advantage of differences in absorbency capacity, such as by placing a lower capacity absorbent material layer closer to the liner 42 and a higher capacity absorbent material closer to the outer cover layer 40. Likewise, discrete portions of an absorbent single-layered structure may encompass higher capacity absorbents, and other discrete portions of the structure may encompass lower capacity absorbents.

As a general rule, the superabsorbent material is present in the absorbent web in an amount of from about 0 to about 90 weight percent based on total weight of the web. The web may have a density within the range of about 0.10 to about 0.60 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and desirably is capable of absorbing more than about 25 times its weight in liquid. Suitable superabsorbent materials are readily available from various suppliers.

After being formed or cut into a desired shape, the absorbent web material may be wrapped or encompassed by a suitable tissue or meltblown web or the like wrap sheet that aids in maintaining the integrity and shape of the absorbent structure 44.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein by reference to the extent they are consistent (i.e., not in conflict) herewith.

In a particular aspect of the absorbent article of the present invention, the absorbent structure 44 may also be elastic. The absorbent structure 44 may include an elastomeric coform absorbent web material. Such materials are described for instance in U.S. Pat. Nos. 6,231,557 B1 and 6,362,389 B1, which are each incorporated by reference herein to the extent they are consistent (i.e., not in conflict) herewith. Other examples of usable elastomeric absorbent bodies are described in international patent application WO 03/051254 and U.S. Pat. Nos. 5,964,743, 5,645,542, 6,231,557, and 6,362,389 B1, each of which are incorporated by reference herein to the extent they are consistent (i.e., not in conflict) herewith.

For purposes of the present invention, the gasket flap 200 surfaces, outer surface 202 and inner surface 204, may be tested to determine the kinetic coefficient of friction between such surfaces. It is believed that the kinetic coefficients of friction are reproducible and measurable, and are representative of actual product use conditions. The kinetic coefficients of friction can be determined using ASTM method D 1894-00. The ASTM procedure is incorporated herein by reference. The ASTM procedure calls for a sample size of 250 mm in the MD and 130 mm in the CD. To measure the coefficient of friction between samples from an actual absorbent article, it may be necessary to modify the ASTM procedure to accommodate the reduced sample sizes. Such modifications may be readily accomplished by those of ordinary skill in the art.

When introducing elements of the present invention or the preferred aspect(s) thereof, the articles “a”, “an”, “the”, and “said” are intended to mean that there are one or more of the elements. The terms “comprising,” “including”, and “having” are intended to be inclusive and mean that there may be additional elements other than the listed elements.

As various changes could be made in the above constructions without departing from the scope of the invention, it is intended that all matter contained in the above description or illustrated in the accompanying drawings shall be interpreted as illustrative and not in a limiting sense.

1. A disposable garment adapted to be worn about a lower torso of a body of a wearer and comprising:
a chassis comprising leg elastics defining a pair of leg openings, and a waistband defining a waist opening spaced from each of the leg openings, wherein the leg openings and the waist opening are collectively referred to as a plurality of garment openings;
a gasket flap located at one of the plurality of garment openings, wherein the gasket flap comprises:
a body-facing surface having a first coefficient of friction and an inner surface that has a second coefficient of friction different from that of the bodyfacing surface, and
a first end and a second end bonded to the garment with a graduated bond to increase resistance against roll back of the gasket flap as the inner surface is exposed.

2. The disposable garment of claim 1 wherein the graduated bond is a permanent bond.

3. The disposable garment of claim 2 wherein the gasket flap has a free edge having a slit therein to encourage roll back of the gasket flap.

4. The disposable garment of claim 1 wherein a pair of the gasket flaps are configured in series.

5. The disposable garment of claim 1 wherein a pair of the gasket flaps are configured in parallel.

6. The disposable garment of claim 1 wherein the gasket flap is positioned at an area of the garment that when worn, contacts a body region of the wearer selected from the group consisting of a back region, a stomach region, and a waist region.

7. The disposable garment of claim 1 wherein the gasket flap is positioned at an area of the garment that when worn, that contacts a body region of the wearer selected from the group consisting of a thigh region and a leg region.

8. The disposable garment of claim 1 wherein the gasket flap is positioned at an area of the garment that when worn, contacts a body region of the wearer, selected from the group consisting of a buttocks region and a hip region.

9. The disposable garment of claim 1 wherein the gasket flap has a surface area and a gradient coefficient of friction over the surface area.

10. The disposable garment of claim 1 wherein the gasket flap has a surface area and a substantially uniform coefficient of friction over the surface area.

11. The disposable garment of claim 1 wherein the gasket flap comprises a coating.

12. The disposable garment of claim 1 further including a lip on a free edge of the gasket flap.

13. The disposable garment of claim 1 wherein the gasket flap comprises an elastomeric material.

14. The disposable garment of claim 1 wherein the first coefficient of friction is less than the second coefficient of friction.

15. The disposable garment of claim 1 wherein the graduated bond comprises a continuous bond that has a shape that widens in the lateral direction as it extends in the longitudinal direction toward the waist region.

16. The disposable garment of claim 1 wherein the graduated bond comprises a discontinuous bond of discrete bond points, each of the discrete bond points increase in peel strength in the longitudinal direction toward the waist region.

17. The disposable garment of claim 16 wherein the discrete bond points are substantially the same in size.

18. The disposable garment of claim 1 wherein the graduated bond comprises a discontinuous bond of discrete bond points, each of the discrete bond points having substantially the same peel strength in the longitudinal direction toward the waist region.

19. The disposable garment of claim 18 where the discrete bond points differ in size.

20. The disposable garment of claim 1 wherein the graduated bond is continuous in area and exhibits a physical property strength gradient.

21. A garment adapted to be worn about a lower torso of a body of a wearer and comprising:
a chassis comprising a front region, a back region, a crotch region positioned between the first region and the back region, and a waist region formed at the upper portion of the front region and the back region;
a pair of side panels adapted to overlie a hip area of the wearer and thereby form a pair of laterally spaced leg openings and a waistband defining a waist opening spaced from each of the leg openings, wherein the leg openings and the waist opening are collectively referred to as a plurality of garment openings;
a gasket flap at one of the plurality of garment openings, wherein the gasket flap comprises:
a body-facing surface having a first coefficient of friction and an inner surface that has a second coefficient of friction different from that of the bodyfacing surface, and
a first end and a second end bonded to the garment with a graduated bond to increase resistance against roll back of the gasket flap as the inner surface is exposed.
